(19) United States
(12) Patent Application Publication
(10) Pub. No.: US 2024/0216245 A1
(43) Pub. Date: Jul. 4, 2024

(54) BODY WASH TO TARGET AND ELIMINATE KEY BACTERIA FOR PROLONGED CONTROL OF BODY ODOR

(71) Applicant: Bradley Cook, Las Vegas, NV (US)

(72) Inventor: Bradley Cook, Las Vegas, NV (US)

(21) Appl. No.: 18/403,088

(22) Filed: Jan. 3, 2024

Related U.S. Application Data
(60) Provisional application No. 63/478,372, filed on Jan. 4, 2023.

(51) Int. Cl. A61K 8/49; A61Q 17/00; A61Q 19/10

(52) U.S. Cl. CPC A61K 8/494; A61Q 17/005; A61Q 19/10; A61K 2800/77

(57) ABSTRACT

An underarm rinse solution composed of water, Hydroxyethyl cellulose, Propanediol, a Sodium Hydroxide and water mixture, Soyethyl Morpholinium Ethosulfate, Cocamidopropyl PG-Dimonium Chloride Phosphate, a Sodium Benzoate and Potassium Sorbate mixture, and a citric acid and water mixture. The solution targets, eliminates, and minimizes growth of Staphylococcus hominis, Staphylococcus epidermidis, and Cutibacterium avidum. The water acts as a carrier and diluent, Hydroxyethylcellulose increases viscosity, Propanediol retains moisture, Sodium Hydroxide adjusts pH, Soyethyl Morpholinium Ethosulfate neutralizes odor, Cocamidopropyl PG-Dimonium Chloride Phosphate conditions skin, Sodium Benzoate and Potassium Sorbate preserve the product, and Citric Acid maintains pH stability. Prolonged use results in reduction of the targeted bacteria and body odor, while maintaining a natural, healthy microbiome.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### BACKGROUND OF THE INVENTION

### Field of the Invention

The present invention relates to a chemical mixture or formulation for control of body odor and, more specifically, to a mixture or formulation which targets a specific bacterial strains for prolonged control of body odor without significant disruption to the natural skin microbiome.

### Description of Related Art

Body odor is a sweat-related odor which manifests on essentially all individuals, is foul-smelling and is pathologic and termed bromhidrosis. Research indicates body odor is caused by Staphylococcus hominis, Staphylococcus epidermidis, and Cutibacterium avidum. Humans have three types of sweat glands: eccrine, apocrine and apocrine sweat glands, as well as sebaceous glands which influence the composition of sweat. Although eccrine sweat glands are the most ubiquitous and are responsible for the largest volume of sweat excretion, bromhidrosis is interaction of apocrine sweat glands with various microorganisms. It's not until the apocrine sweat is metabolized by axillary microorganisms such as Micrococcaceae, Propionibacteria, Staphylococcus and nondiptheiroid Corynebacterium species does it develop its characteristic odor

A major odorant is primarily generated by the disinfectant resistant staphylococcus hominis and is composed of volatile sulfur compounds such as 3-Methyl-3-sulfanylhexanol3-6. These volatile sulfur compounds are secreted by apocrine glands as glycine-cysteine conjugates and are enzymatically cleaved by bacterial dipeptidases and C—S lyases which release odoriferous mercaptoalcohols. Other malodors include the volatile fatty acid 3-methyl-2-hexenoic acid (3M2H), which has a goat-like odor, and 3-methyl-3-hydroxy-hexanoic acid, which has a cumin like odor. Studies indicate that 3M2H is released from the skin surface after interacting with Corynebacterium striatum and Corynebacterium bovis. Another common malodor that is characterized by its vinegar-like smell is caused by Propionibacterium metabolizing glycerol and lactic acid, leading to the production of acetic and propionic acid. Each of the aforementioned compounds, when combined with Staphylococcus hominis, Staphylococcus epidermidis, and Cutibacterium avidum, cause what is commonly known as BO. The composition of the skin microbiota is diverse and can be functionally distinct between individuals. The skin microbiome and the volatile odorants produced are also dynamic and have been shown to shift in certain environments

The skin microbiome contributes to the barrier function of the skin and ensures skin homeostasis. The secretion of protease enzymes by skin microbes is involved in the desquamation process and stratum corneum renewal. Sebum and free fatty acid production are involved in pH regulation. The interruption and depletion of the skin microbiota prevents the skin from an important role which is protecting against potential pathogenic microorganisms by competition and antimicrobial peptide (AMP) production by commensal bacteria or Malassezia fungi. Significant interruption of the skin microbiome produces a range of indoles that inhibit many harmful yeasts and molds. The result of increased yeast and molds causes skin and yeast infections.

Though claiming to remove 99.9% of bacteria, many conventional, commercially available body washes are ineffective at targeting and eliminating these specifically aforementioned bacteria, especially for a significant time span (over 72 hours) and thus require the use of multiple products including deodorants and antiperspirants to reduce or mask the smell caused by the aforementioned bacteria. Additionally, continued and prolonged use of the aforementioned products can lead to disruption and/or deterioration of the natural skin microbiome, which is difficult to restore.

There remains a need in the art for a solution to at least one of the aforementioned problems. Several formulas and products are commonly used to address body odor and/or used which promote a healthy skin microbiome. However, there remains a need for a single formulation used to manage the underlying conditions and causes for body odor without significantly disrupting the skin microbiome.

### SUMMARY OF THE INVENTION

In general, in a first aspect, the subject matter features an underarm rinse solution having water, Hydroxyethyl cellulose, Propanediol, a Sodium Hydroxide and water mixture, Soyethyl Morpholinium Ethosulfate, Cocamidopropyl PG-Dimonium Chloride Phosphate, a Sodium Benzoate and Potassium Sorbate mixture, and a citric acid and water mixture. This underarm rinse solution targets, eliminates and minimizes growth of Staphylococcus hominis, Staphylococcus epidermidis, and Cutibacterium avidum.

Embodiments of the subject matter may include one or more of the following features. The water may be the primary solvent in which all other ingredients are dissolved and may act as a carrier and diluent, ensuring that the ingredients are distributed evenly when the underarm rinse solution is applied to the skin and facilitates the interaction between the purifying ingredients and the bacteria on the skin. The Hydroxyethylcellulose may be a plant-derived thickening agent for increasing the viscosity of the rinse, ensuring that the body rinse adheres to the skin long enough for the other ingredients to work and helps in providing a smooth application experience and also allows for a uniform distribution of ingredients during application. The Propanediol, usually derived from corn, may be a humectant and solvent and helps retain moisture, ensuring that the skin remains hydrated and not dried out after the underarm rinse solution is washed off.

The water with Sodium Hydroxide mixture may be used to adjust the pH of the product ensuring the underarm rinse solution remains gentle on the skin while still being effective. Sodium hydroxide in the right proportions ensures that the underarm rinse solution maintains an optimal pH level conducive to bacterial removal. The Soyethyl Morpholinium Ethosulfate may be an emulsifier which helps in neutralizing odor and removing or washing away bacteria, including the Staphylococcus hominis and ensures that the underarm rinse solution effectively targets the specific underarm malodor. The Cocamidopropyl PG-Dimonium Chloride Phosphate may be a compound which acts as a conditioning agent, making the skin feel soft and smooth and acts as a gentle surfactant that possesses purifying properties, enhancing the underarm rinse solution efficacy in removing bacteria and ensuring that the skin is free from odor and feels good post-rinse.

The Sodium Benzoate and Potassium Sorbate mixture may ensure that the product remains free from harmful microbes and has a longer shelf life wherein the sodium benzoate and potassium sorbate are preservatives. The Citric Acid may be used to adjust the pH of the formulation, acting as a buffering agent, ensuring that the pH remains stable over time and wherein a balanced pH maintains the efficacy of the body rinse and ensures skin compatibility. Prolonged use may result in reduction of the Staphylococcus hominis, Staphylococcus epidermidis, and Cutibacterium avidum and reduction of body odor. A natural, healthy microbiome may be maintained.

In a first implementation of the invention, presented is a novel formula to ensure the decrease and elimination of the specific bacterium that cause body odor, and does so for an extended period of time with regular use. The formula is an all-in-one body wash containing several conventional ingredients, formulated is such a manner as to uniquely work together to efficiently ensure the targeting and elimination of body odor causing bacteria and microbes while maintaining the health of the naturally occurring skin biome.

In the first implementation of the invention, an aqueous solution of Benzoyl Peroxide, Aloe Vera, Glycolic Acid, Glycerin, Cocamidopropyl Betaine (CAPB), Vitamin E, Gluconolactone, Sodium Benzoate, and Citric Acid are formulated. The formulation of these ingredients serve to address issues of body odor and bacterial growth while maintaining the natural skin microbiome.

### DETAILED DESCRIPTION OF THE INVENTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The terms bacteria and microbes are used interchangeably to mean a biological system outside the normal human microbiome to the instant invention.

The term microbiome as used in the specification is meant to mean either a normal or natural human skin biological system.

The subject matter disclosed herein relates to an underarm rinse solution. This solution is a composition that includes several components, each serving a specific purpose. The primary solvent in the solution is water. It acts as a carrier and diluent, ensuring that the ingredients are distributed evenly when the underarm rinse solution is applied to the skin and facilitates the interaction between the purifying ingredients and the bacteria on the skin.

One of the components of the solution is Hydroxyethyl cellulose, a plant-derived thickening agent. It increases the viscosity of the rinse, ensuring that the body rinse adheres to the skin long enough for the other ingredients to work. It also provides a smooth application experience and allows for a uniform distribution of ingredients during application.

Another component is Propanediol, which is usually derived from corn. It acts as a humectant and solvent and helps retain moisture, ensuring that the skin remains hydrated and not dried out after the underarm rinse solution is washed off.

The solution also includes a mixture of Sodium Hydroxide and water. This mixture is used to adjust the pH of the product, ensuring the underarm rinse solution remains gentle on the skin while still being effective. Sodium hydroxide in the right proportions ensures that the underarm rinse solution maintains an optimum pH level conducive to bacterial removal.

Soyethyl Morpholinium Ethosulfate is another component of the solution. It is an emulsifier which helps in neutralizing odor and removing or washing away bacteria, including Staphylococcus hominis. It ensures that the underarm rinse solution effectively targets the specific underarm malodor.

The solution also includes Cocamidopropyl PG-Dimonium Chloride Phosphate, a compound which acts as a conditioning agent. It makes the skin feel soft and smooth and acts as a gentle surfactant that possesses purifying properties, enhancing the underarm rinse solution efficacy in removing bacteria and ensuring that the skin is free from odor and feels good post-rinse.

A mixture of Sodium Benzoate and Potassium Sorbate is also included in the solution. These ingredients ensure that the product remains free from harmful microbes and has a longer shelf life. They are preservatives that help maintain the quality of the solution over time.

The solution also includes a mixture of citric acid and water. Citric Acid is used to adjust the pH of the formulation, acting as a buffering agent. It ensures that the pH remains stable over time. A balanced pH maintains the efficacy of the body rinse and ensures skin compatibility.

The underarm rinse solution is designed to target, eliminate, and minimize the growth of specific bacteria causing underarm odor, including Staphylococcus hominis, Staphylococcus epidermidis, and Cutibacterium avidum. Prolonged use of the solution results in a reduction of these bacteria and a reduction of body odor. Furthermore, the solution is formulated to maintain a natural, healthy microbiome.

In the underarm rinse solution, water serves a pivotal role as the primary solvent. It acts as a carrier and diluent for the other ingredients, ensuring that they are evenly distributed when the solution is applied to the skin. This is particularly beneficial as it facilitates the interaction between the purifying ingredients and the bacteria on the skin. In some cases, the water may be sourced from various origins, such as tap water, distilled water, or purified water, among others. The amount of water in the solution can also vary, depending on the desired consistency and concentration of the other ingredients. In some cases, the water content may be between about 50% and 99% by weight of the total solution, between about 60% and 90%, between about 70% and 80%, or any other suitable range. This flexibility in the water content allows for the adjustment of the solution's properties to suit different user preferences or application conditions.

Hydroxyethyl cellulose, a component of the underarm rinse solution, is a plant-derived thickening agent. It serves to increase the viscosity of the rinse, ensuring that the solution adheres to the skin long enough for the other ingredients to work. This adherence is particularly beneficial as it allows for a uniform distribution of ingredients during application, providing a smooth application experience. The Hydroxyethyl cellulose can be derived from various plant sources, such as cotton or wood pulp, among others. The amount of Hydroxyethyl cellulose in the solution can also vary, depending on the desired viscosity and adherence properties of the solution. In some cases, the Hydroxyethyl cellulose content may be between about 0.1% and 5% by weight of the total solution, between about 0.5% and 3%, between about 1% and 2%, or any other suitable range. This flexibility in the Hydroxyethyl cellulose content allows for the adjustment of the solution's properties to suit different user preferences or application conditions.

Propanediol, another component of the underarm rinse solution, is typically derived from corn. It serves as a humectant and solvent in the solution, playing a pivotal role in retaining moisture. This is particularly beneficial as it ensures that the skin remains hydrated and not dried out after the underarm rinse solution is washed off. The Propanediol can be derived from various sources, such as corn, but in some cases, it may also be derived from other plant sources or synthesized. The amount of Propanediol in the solution can also vary, depending on the desired moisturizing properties of the solution. In some cases, the Propanediol content may be between about 1% and 20% by weight of the total solution, between about 2% and 15%, between about 3% and 10%, or any other suitable range. This flexibility in the Propanediol content allows for the adjustment of the solution's properties to suit different user preferences or application conditions.

The underarm rinse solution also incorporates a mixture of Sodium Hydroxide and water. This mixture plays a role in adjusting the pH of the product. Maintaining an appropriate pH level is particularly beneficial as it ensures the underarm rinse solution remains gentle on the skin while still being effective. Sodium Hydroxide, when used in the right proportions, ensures that the underarm rinse solution maintains an optimum pH level conducive to bacterial removal. The Sodium Hydroxide can be sourced from various suppliers and the amount used can vary, depending on the desired pH level of the solution. In some cases, the Sodium Hydroxide content may be between about 0.1% and 2% by weight of the total solution, between about 0.2% and 1.5%, between about 0.3% and 1%, or any other suitable range. This flexibility in the Sodium Hydroxide content allows for the adjustment of the solution's properties to suit different user preferences or application conditions.

Soyethyl Morpholinium Ethosulfate is another component of the underarm rinse solution. It serves as an emulsifier in the solution, playing a pivotal role in neutralizing odor and removing or washing away bacteria, including Staphylococcus hominis. This is particularly beneficial as it ensures that the underarm rinse solution effectively targets the specific underarm malodor. The Soyethyl Morpholinium Ethosulfate can be sourced from various suppliers and the amount used can vary, depending on the desired odor-neutralizing properties of the solution. In some cases, the Soyethyl Morpholinium Ethosulfate content may be between about 0.1% and 5% by weight of the total solution, between about 0.5% and 3%, between about 1% and 2%, or any other suitable range. This flexibility in the Soyethyl Morpholinium Ethosulfate content allows for the adjustment of the solution's properties to suit different user preferences or application conditions.

Cocamidopropyl PG-Dimonium Chloride Phosphate is another component of the underarm rinse solution. It serves as a conditioning agent and a gentle surfactant in the solution. As a conditioning agent, it contributes to making the skin feel soft and smooth after the rinse. As a surfactant, it possesses purifying properties, enhancing the underarm rinse solution's efficacy in removing bacteria and ensuring that the skin is free from odor and feels good post-rinse. The Cocamidopropyl PG-Dimonium Chloride Phosphate can be sourced from various suppliers and the amount used can vary, depending on the desired conditioning and purifying properties of the solution. In some cases, the Cocamidopropyl PG-Dimonium Chloride Phosphate content may be between about 0.1% and 5% by weight of the total solution, between about 0.5% and 3%, between about 1% and 2%, or any other suitable range. This flexibility in the Cocamidopropyl PG-Dimonium Chloride Phosphate content allows for the adjustment of the solution's properties to suit different user preferences or application conditions.

The underarm rinse solution also includes a mixture of Sodium Benzoate and Potassium Sorbate. These ingredients serve as preservatives in the solution, ensuring that the product remains free from harmful microbes and has a longer shelf life. This is particularly beneficial as it helps maintain the quality of the solution over time. The Sodium Benzoate and Potassium Sorbate can be sourced from various suppliers and the amount used can vary, depending on the desired preservative properties of the solution. In some cases, the Sodium Benzoate and Potassium Sorbate content may be between about 0.1% and 2% by weight of the total solution, between about 0.2% and 1.5%, between about 0.3% and 1%, or any other suitable range. This flexibility in the Sodium Benzoate and Potassium Sorbate content allows for the adjustment of the solution's properties to suit different user preferences or application conditions.

The underarm rinse solution also incorporates a mixture of citric acid and water. Citric Acid plays a pivotal role in adjusting the pH of the formulation, acting as a buffering agent. Maintaining a stable pH is particularly beneficial as it ensures that the efficacy of the body rinse is maintained and ensures skin compatibility. The Citric Acid can be sourced from various suppliers and the amount used can vary, depending on the desired pH level of the solution. In some cases, the Citric Acid content may be between about 0.1% and 2% by weight of the total solution, between about 0.2% and 1.5%, between about 0.3% and 1%, or any other suitable range. This flexibility in the Citric Acid content allows for the adjustment of the solution's properties to suit different user preferences or application conditions.

The underarm rinse solution incorporates ingredients that both target the specific bacteria causing underarm odor and ensure a smooth, skin-friendly application. The solution is designed to target, eliminate, and minimize the growth of specific bacteria causing underarm odor, including Staphylococcus hominis, Staphylococcus epidermidis, and Cutibacterium avidum. This is achieved through the inclusion of specific ingredients such as Soyethyl Morpholinium Ethosulfate, an emulsifier that helps in neutralizing odor and removing or washing away bacteria, including Staphylococcus hominis. It ensures that the underarm rinse solution effectively targets the specific underarm malodor.

Furthermore, the solution is formulated to ensure a smooth, skin-friendly application. This is achieved through the inclusion of Hydroxyethyl cellulose, a plant-derived thickening agent that increases the viscosity of the rinse, ensuring that the body rinse adheres to the skin long enough for the other ingredients to work. It also provides a smooth application experience and allows for a uniform distribution of ingredients during application. Additionally, Propanediol, which is usually derived from corn, acts as a humectant and solvent and helps retain moisture, ensuring that the skin remains hydrated and not dried out after the underarm rinse solution is washed off.

Moreover, Cocamidopropyl PG-Dimonium Chloride Phosphate, a compound which acts as a conditioning agent, makes the skin feel soft and smooth and acts as a gentle surfactant that possesses purifying properties, enhancing the underarm rinse solution efficacy in removing bacteria and ensuring that the skin is free from odor and feels good post-rinse. This combination of ingredients ensures that the underarm rinse solution not just targets the specific bacteria causing underarm odor but also ensures a smooth, skin-friendly application.

With prolonged use, the underarm rinse solution is designed to result in a reduction of specific bacteria causing underarm odor, including Staphylococcus hominis, Staphylococcus epidermidis, and Cutibacterium avidum. This reduction in bacteria is particularly beneficial as it directly contributes to a reduction of body odor. The solution achieves this through the combined action of its various components. For instance, Soyethyl Morpholinium Ethosulfate, an emulsifier in the solution, helps in neutralizing odor and removing or washing away bacteria, including Staphylococcus hominis. This ensures that the underarm rinse solution effectively targets the specific underarm malodor. Additionally, Cocamidopropyl PG-Dimonium Chloride Phosphate, a compound in the solution, acts as a gentle surfactant that possesses purifying properties, enhancing the underarm rinse solution's efficacy in removing bacteria and ensuring that the skin is free from odor.

Furthermore, the solution's pH level, adjusted by the mixture of Sodium Hydroxide and water and the mixture of citric acid and water, is maintained at an optimum level conducive to bacterial removal. This pH level ensures that the underarm rinse solution remains gentle on the skin while still being effective in its action against bacteria. Over time, with consistent use, this results in a reduction of the Staphylococcus hominis, Staphylococcus epidermidis, and Cutibacterium avidum populations on the skin, thereby reducing body odor.

It is also worth noting that the underarm rinse solution is formulated to maintain a natural, healthy microbiome. This means that while it targets and reduces the specific bacteria causing underarm odor, it does not adversely affect the overall skin microbiome. This balance between effective odor control and maintaining a healthy skin microbiome is a notable aspect of the underarm rinse solution, particularly with prolonged use.

Another notable aspect of the underarm rinse solution is its ability to maintain a natural, healthy microbiome. The skin is home to a diverse community of microorganisms, collectively known as the skin microbiome. This microbiome plays a pivotal role in skin health, including protection against pathogens and the regulation of immune responses. Therefore, maintaining a balanced and healthy skin microbiome is of utmost relevance in skincare products.

The underarm rinse solution is formulated in such a way that while it targets and reduces the specific bacteria causing underarm odor, it does not adversely affect the overall skin microbiome. This is achieved through the careful selection and combination of its components. For instance, Propanediol, a component of the solution, acts as a humectant and helps retain moisture, ensuring that the skin remains hydrated. This is particularly beneficial as it helps maintain the natural balance of the skin microbiome, which thrives in a moist environment.

Furthermore, the pH level of the solution, adjusted by the mixture of Sodium Hydroxide and water and the mixture of citric acid and water, is maintained at an optimum level conducive not just to bacterial removal but also to the maintenance of a healthy skin microbiome. A balanced pH is particularly beneficial as it supports the growth and survival of beneficial skin microorganisms while inhibiting the growth of harmful ones.

Moreover, the solution includes Cocamidopropyl PG-Dimonium Chloride Phosphate, a compound that acts as a gentle surfactant. This compound possesses purifying properties, enhancing the underarm rinse solution's efficacy in removing bacteria. However, its gentle nature ensures that it does not disrupt the overall skin microbiome, thereby maintaining a healthy balance of skin microorganisms.

Through these mechanisms, the underarm rinse solution ensures that while it effectively controls underarm odor by targeting specific odor-causing bacteria, it also maintains a natural, healthy microbiome. This balance between effective odor control and maintaining a healthy skin microbiome is a notable aspect of the underarm rinse solution, particularly with prolonged use.

One formulation is an underarm rinse solution comprising: water; Hydroxyethyl cellulose, Propanediol, a Sodium Hydroxide and water mixture, Soyethyl Morpholinium Ethosulfate, Cocamidopropyl PG-Dimonium Chloride Phosphate, a Sodium Benzoate and Potassium Sorbate mixture, and a citric acid and water mixture wherein the underarm rinse solution targets, eliminates and minimizes growth of Staphylococcus hominis, Staphylococcus epidermidis, and Cutibacterium avidum.

The water is the primary solvent in which all other ingredients are dissolved and the water acts as a carrier and diluent, ensuring that the ingredients are distributed evenly when the underarm rinse solution is applied to the skin and facilitates the interaction between the purifying ingredients and the bacteria on the skin.

The Hydroxyethylcellulose is a plant-derived thickening agent for increasing the viscosity of the rinse, ensuring that the body rinse adheres to the skin long enough for the other ingredients to work and helps in providing a smooth application experience and also allows for a uniform distribution of ingredients during application.

The Propanediol is derived usually from corn and is a humectant and solvent and helps retain moisture, ensuring that the skin remains hydrated and not dried out after the underarm rinse solution is washed off.

The water with Sodium Hydroxide mixture is used to adjust the pH of the product ensuring the underarm rinse solution remains gentle on the skin while still being effective. Sodium hydroxide in the right proportions ensures that the underarm rinse solution maintains an optimal pH level conducive to bacterial removal.

The Soyethyl Morpholinium Ethosulfate is an emulsifier which helps in neutralizing odor and removing or washing away bacteria, including the Staphylococcus hominis and ensures that the underarm rinse solution effectively targets the specific underarm malodor.

The Cocamidopropyl PG-Dimonium Chloride Phosphate is a compound which acts as a conditioning agent, making the skin feel soft and smooth and acts as a gentle surfactant that possesses purifying properties, enhancing the underarm rinse solution efficacy in removing bacteria and ensuring that the skin is free from odor and feels good post-rinse.

The Sodium Benzoate and Potassium Sorbate mixture ensure that the product remains free from harmful microbes and has a longer shelf life wherein the sodium benzoate and potassium sorbate are preservatives.

The Citric Acid is used to adjust the pH of the formulation, acting as a buffering agent, ensuring that the pH remains stable over time and wherein a balanced pH maintains the efficacy of the body rinse and ensures skin compatibility.

Prolonged use results in reduction of the Staphylococcus hominis, Staphylococcus epidermidis, and Cutibacterium avidum and reduction of body odor and a natural, healthy microbiome is maintained.

Other ingredients which may be of benefit in the body rinse solution are described as follows:

In the body rinse solution, Benzoyl Peroxide may play a role. It is a well-known antibacterial agent, which is particularly effective against the bacteria Staphylococcus hominis, Staphylococcus epidermidis, and Cutibacterium avidum. These bacteria are known contributors to body odor, and the antibacterial action of Benzoyl Peroxide helps to control and reduce their growth, thereby helping to control and reduce body odor.

The concentration of Benzoyl Peroxide in the body rinse solution can be adjusted according to the specific requirements of the user or the specific application. In some cases, a higher concentration of Benzoyl Peroxide may be used for a stronger antibacterial effect. In other cases, a lower concentration may be used, depending on the sensitivity of the user's skin or the desired level of odor control. It is also possible to adjust the concentration of Benzoyl Peroxide in combination with the other ingredients in the body wash composition, to achieve a balanced effect of antibacterial action, skin cleansing, and maintenance of a healthy microbiome.

Aloe Vera may be another ingredient in the body rinse solution. It is known for its soothing and moisturizing properties. In the context of the body wash, Aloe Vera serves to soothe the skin and provide moisture, which can be particularly beneficial after the cleansing action of the other ingredients. This can help to maintain the health and integrity of the skin, which is an integral part of maintaining a healthy microbiome.

The concentration of Aloe Vera in the body rinse solution can be adjusted according to the specific requirements of the user or the specific application. In some cases, a higher concentration of Aloe Vera may be used for its soothing and moisturizing properties. In other cases, a lower concentration may be used, depending on the sensitivity of the user's skin or the desired level of skin hydration. It is also possible to adjust the concentration of Aloe Vera in combination with the other ingredients in the body wash composition, to achieve a balanced effect of skin cleansing, antibacterial action, and maintenance of a healthy microbiome.

Glycolic Acid may be another component of the body rinse solution. It is an alpha-hydroxy acid that is known for its exfoliating properties. In the context of the body rinse, Glycolic Acid serves to exfoliate the skin, removing dead skin cells and promoting the renewal of the skin surface. This can contribute to the overall cleanliness of the skin and can also help to enhance the effectiveness of the other ingredients in the body rinse solution, by facilitating their penetration into the skin.

The concentration of Glycolic Acid in the body rinse solution can be adjusted according to the specific requirements of the user or the specific application. In some cases, a higher concentration of Glycolic Acid may be used for a stronger exfoliating effect. In other cases, a lower concentration may be used, depending on the sensitivity of the user's skin or the desired level of skin exfoliation. It is also possible to adjust the concentration of Glycolic Acid in combination with the other ingredients in the body wash composition, to achieve a balanced effect of skin exfoliation, antibacterial action, skin cleansing, and maintenance of a healthy microbiome.

Glycerin may be another component of the body rinse solution. It is a humectant, which means it has the ability to attract and retain moisture. In the context of the body wash, Glycerin serves to hydrate the skin, helping to maintain its moisture balance during and after cleansing. This can contribute to the overall health and comfort of the skin, which is an integral part of maintaining a healthy microbiome.

The concentration of Glycerin in the body rinse solution can be adjusted according to the specific requirements of the user or the specific application. In some cases, a higher concentration of Glycerin may be used for its hydrating properties. In other cases, a lower concentration may be used, depending on the sensitivity of the user's skin or the desired level of skin hydration. It is also possible to adjust the concentration of Glycerin in combination with the other ingredients in the body rinse solution, to achieve a balanced effect of skin hydration, antibacterial action, skin cleansing, and maintenance of a healthy microbiome.

Cocamidopropyl Betaine (CAPB) may be another component of the body rinse solution. It is a surfactant, which means it has the ability to lower the surface tension between two liquids or between a liquid and a solid. In the context of the body rinse, CAPB serves to facilitate the removal of dirt, oils, and other impurities from the skin. It does this by allowing water to mix with these impurities, enabling them to be washed away. This contributes to the overall cleanliness of the skin, which is an integral part of maintaining a healthy microbiome.

The concentration of Cocamidopropyl Betaine (CAPB) in the body rinse solution can be adjusted according to the specific requirements of the user or the specific application. In some cases, a higher concentration of CAPB may be used for its cleansing properties. In other cases, a lower concentration may be used, depending on the sensitivity of the user's skin or the desired level of skin cleansing. It is also possible to adjust the concentration of CAPB in combination with the other ingredients in the body rinse solution, to achieve a balanced effect of skin cleansing, antibacterial action, and maintenance of a healthy microbiome.

Vitamin E may be another component of the body rinse solution. It is known for its antioxidant properties. In the context of the body wash, Vitamin E serves to protect the skin from damage caused by free radicals. This can contribute to the overall health and integrity of the skin, which is an integral part of maintaining a healthy microbiome.

The concentration of Vitamin E in the body rinse solution can be adjusted according to the specific requirements of the user or the specific application. In some cases, a higher concentration of Vitamin E may be used for its antioxidant properties. In other cases, a lower concentration may be used, depending on the sensitivity of the user's skin or the desired level of skin protection. It is also possible to adjust the concentration of Vitamin E in combination with the other ingredients in the body wash composition, to achieve a balanced effect of skin protection, antibacterial action, skin cleansing, and maintenance of a healthy microbiome.

Gluconolactone may be another component of the body rinse solution. It is a polyhydroxy acid known for its gentle exfoliating properties. In the context of the body wash, Gluconolactone serves to gently exfoliate the skin, promoting the removal of dead skin cells and enhancing the renewal of the skin surface. This can contribute to the overall cleanliness of the skin and can also help to enhance the effectiveness of the other ingredients in the body wash composition, by facilitating their penetration into the skin.

The concentration of Gluconolactone in the body rinse solution can be adjusted according to the specific requirements of the user or the specific application. In some cases, a higher concentration of Gluconolactone may be used for a stronger exfoliating effect. In other cases, a lower concentration may be used, depending on the sensitivity of the user's skin or the desired level of skin exfoliation. It is also possible to adjust the concentration of Gluconolactone in combination with the other ingredients in the body wash composition, to achieve a balanced effect of skin exfoliation, antibacterial action, skin cleansing, and maintenance of a healthy microbiome.

Sodium Benzoate may be another component of the body rinse solution. It is a type of salt that is commonly used as a preservative in personal care products. In the context of the body wash, Sodium Benzoate serves to preserve the integrity of the other ingredients, preventing their degradation over time. This can contribute to the overall stability and shelf-life of the body wash, ensuring that it remains effective even after prolonged storage.

The concentration of Sodium Benzoate in the body rinse solution can be adjusted according to the specific requirements of the user or the specific application. In some cases, a higher concentration of Sodium Benzoate may be used for its preservative properties. In other cases, a lower concentration may be used, depending on the desired shelf-life of the body rinse or the sensitivity of the user's skin. It is also possible to adjust the concentration of Sodium Benzoate in combination with the other ingredients in the body wash composition, to achieve a balanced effect of preservation, antibacterial action, skin cleansing, and maintenance of a healthy microbiome.

Citric Acid may be another component of the body rinse solution. It is a naturally occurring alpha-hydroxy acid known for its ability to adjust the pH of skincare products. In the context of the body rinse, Citric Acid serves to maintain the PH balance of the composition, ensuring that it is suitable for use on the skin. A balanced pH can contribute to the overall effectiveness of the body wash, as it can help to maintain the health and integrity of the skin's natural acid mantle, which is an integral part of maintaining a healthy microbiome.

The concentration of Citric Acid in the body rinse solution can be adjusted according to the specific requirements of the user or the specific application. In some cases, a higher concentration of Citric Acid may be used for its pH balancing properties. In other cases, a lower concentration may be used, depending on the desired pH of the body rinse or the sensitivity of the user's skin. It is also possible to adjust the concentration of Citric Acid in combination with the other ingredients in the body wash composition, to achieve a balanced effect of pH adjustment, antibacterial action, skin cleansing, and maintenance of a healthy microbiome.

The body wash incorporates ingredients that both target the specific bacteria causing underarm odor and ensure a smooth, skin-friendly application. The ingredients collectively work together to offer a product that can potentially replace conventional deodorants. The use of solvents, thickeners, preservatives, conditioning agents, and pH adjusters allows the product to be effective, safe, and user-friendly.

The balance of ingredients offers the following:
1. Optimal Efficacy: Each ingredient in the formulation plays a specific role. While some directly target bacteria removal, others ensure that the product is applied smoothly and stays on the skin long enough to be effective. If the balance is skewed, the product might either not work effectively or it might not adhere well to the skin, reducing its effectiveness.
2. Safety and Tolerance: Personal care products are applied directly to the skin. An incorrect balance, especially of potent ingredients like preservatives or pH adjusters (like Sodium Hydroxide and Citric Acid), could lead to skin irritation or adverse reactions. Balancing them ensures that they achieve their intended function without causing harm.
3. Physical and Chemical Stability: Over time, products can degrade if their ingredients aren't balanced. For example, without the right preservatives (like Sodium Benzoate and Potassium Sorbate) in the correct amounts, the rinse might become susceptible to microbial growth. On the other hand, too much of these preservatives might compromise skin compatibility.
4. Product Experience: Beyond efficacy and safety, the balance of ingredients also affects the sensory attributes of the product. The viscosity, texture, and feel of the rinse on the skin are determined by ingredients like Hydroxyethylcellulose and Propanediol. An imbalance could result in a product that feels too thick, too runny, or leaves an unpleasant residue.
5. pH Balance: The skin has a natural PH balance that is mildly acidic, generally ranging between 4.5 to 5.5. It's essential that personal care products, especially those meant for direct skin application, respect this pH range to prevent skin disturbances and ensure efficacy. Ingredients like Sodium Hydroxide and Citric Acid help in pH adjustment, and their balanced inclusion ensures the product's pH aligns well with the skin's natural pH.
6. Synergistic Action: Some ingredients, when used together in the right proportions, can enhance the effect of each other. This synergy ensures that the combined effect of the ingredients is greater than the sum of their individual effects. The preservative agents in the rinse, for instance, might work even better in the presence of certain solvents or conditioners.
7. Consistency and Production: From a manufacturing standpoint, maintaining a consistent balance of ingredients ensures that each batch of the product offers the same efficacy, safety, and user experience. It also aids in streamlining the production process and ensuring quality control.

The body wash is a finely tuned orchestration that takes into account science, skin biology, user experience, and production logistics.

Since many modifications, variations, and changes in detail can be made to the described embodiments of the invention, it is intended that all matters in the foregoing description be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. An underarm rinse solution comprising:
water;
Hydroxyethyl cellulose;
Propanediol;
a Sodium Hydroxide and water mixture;
Soyethyl Morpholinium Ethosulfate;
Cocamidopropyl PG-Dimonium Chloride Phosphate;
a Sodium Benzoate and Potassium Sorbate mixture; and
a citric acid and water mixture;
wherein the underarm rinse solution targets, eliminates and minimizes growth of Staphylococcus hominis, Staphylococcus epidermidis, and Cutibacterium avidum.

2. The underarm rinse solution of claim 1 wherein the water is the primary solvent in which all other ingredients are dissolved and the water acts as a carrier and diluent, ensuring that the ingredients are distributed evenly when the underarm rinse solution is applied to the skin and facilitates the interaction between the purifying ingredients and the bacteria on the skin.

3. The underarm rinse solution of claim 1, wherein the Hydroxyethylcellulose increases the viscosity of the rinse, ensuring that the body rinse adheres to the skin long enough for the other ingredients to work and helps in providing a smooth application experience and allows for a uniform distribution of ingredients during application.

4. The underarm rinse solution of claim 1, wherein the Propanediol helps retain moisture, ensuring that the skin remains hydrated and not dried out after the underarm rinse solution is washed off.

5. The underarm rinse solution of claim 1, wherein the water with Sodium Hydroxide mixture is used to adjust the pH of the underarm rinse solution ensuring the underarm rinse solution remains gentle on the skin while still being effective.

6. The underarm rinse solution of claim 1, wherein the Soyethyl Morpholinium Ethosulfate is an emulsifier for neutralizing odor and removing or washing away bacteria, including the Staphylococcus hominis and ensuring that the underarm rinse solution effectively targets the specific underarm malodor.

7. The underarm rinse solution of claim 1, wherein the Cocamidopropyl PG-Dimonium Chloride Phosphate is a conditioning agent for making the skin feel soft and smooth and acting as a gentle surfactant that possesses purifying properties, enhancing the underarm rinse solution efficacy in removing bacteria and ensuring that the skin is free from odor and feels good post-rinse.

8. The underarm rinse solution of claim 1, wherein the Sodium Benzoate and Potassium Sorbate mixture ensures that the underarm rinse solution remains free from harmful microbes and has a longer shelf life.

9. The underarm rinse solution of claim 1, wherein the Citric Acid is used to adjust the pH of the formulation, acting as a buffering agent, ensuring that the pH remains stable over time wherein a balanced pH maintains the efficacy of the body rinse and ensures skin compatibility.

10. The underarm rinse solution of claim 1, wherein prolonged use results in reduction of the Staphylococcus hominis, Staphylococcus epidermidis, and Cutibacterium avidum and reduction of body odor.

11. The underarm rinse solution of claim 1, wherein a natural, healthy microbiome is maintained.
